(19) United States
(12) Patent Application Publication
(10) Pub. No.: US 2022/0330955 A1
(43) Pub. Date: Oct. 20, 2022

(54) A GUIDE PLATE STRUCTURE FOR SPINE SURGERY AND THE PRODUCTION METHOD AND USAGE THEREOF

(71) Applicant: The Second Affiliated Hospital and Yuying Children's Hospital of Wenzhou Medical University, Wenzhou (CN)

(72) Inventors: Haiming Jin, Wenzhou (CN); Xiangyang Wang, Wenzhou (CN); Weimin Zeng, Wenzhou (CN); Xuyao Han, Wenzhou (CN); Yingjing Chen, Wenzhou (CN); Sunren Sheng, Wenzhou (CN); Naifeng Tian, Wenzhou (CN); Sheng Wang, Wenzhou (CN)

(73) Assignee: The Second Affiliated Hospital and Yuying Children's Hospital of Wenzhou Medical University

(21) Appl. No.: 17/232,156

(22) Filed: Apr. 16, 2021

(51) Int. Cl. A61B 17/17; A61B 34/10

(52) U.S. Cl. CPC A61B 17/1757; A61B 34/10; A61B 2017/00526

(57) ABSTRACT

A guide plate for spine surgery incl odes a guide plate body. The guide plate body includes a medial surface matching the back surface of surgical segment centrum. The guide plate body is provided with a guide hole. The outer surface of guide block is conical, the guide plate body is provided with a drill hole corresponding to the guide block. The drill hole matches the outer surface of front end of guide block, the guide block is embedded in the drill hole.

### BACKGROUND OF INVENTION

### 1. Field of the Invention

In spinal operation, the internal fixation of posterior pedicle screw is undoubtedly the bottommost operation technique. However, the accurate insertion of screw is always a difficulty, it depends on the variation of anatomic form, the size and direction of pedicle of vertebral arch. Especially for the patients with deformity of cervical vertebrae, thoracic vertebrae and even spine, it has a complex structure and individual difference, so that the structure has high variability, the manual screw placement without an auxiliary navigation tool has higher risk. It is reported in foreign documents that the error rate of manual placement of pedicle screw is 10%-40%, that is likely to induce serious vascular and nerve injuries.

Therefore, a patient individuation-based 3D printing rapid phototyping technology is used to assist the insertion of pedicle screw. For lower price and simple operation, the technology has been improved and popularized to pedicle screw fixation in different regions of spine.

The rapid phototyping navigation template technology was first used by Radermacher et al. in the research on lumbar pedicle screw placement in 1998. Afterwards, the technology was popularized and improved. Now it has been extensively used in the research on cervical vertebrae, thoracic vertebrae, lumbar and more complex atlantoaxial and lateral curvature screw placement.

In the use of common spinal operation guide plate, for the patient or region with high hone mineral density and hard posterior cortical bone of centrum, the guide pin is hard to break through the corresponding cortical bone of centrum, the guide pin is likely to skid on the surface of untreated cortical bone, leading to inaccurate localization, increasing the difficulty in insertion of guide pin, even failing insertion. The existing guide plate fabrication accuracy is insufficient, the guide pin insertion accuracy cannot be guaranteed, and the insertion of hollow screw is influenced.

### SUMMARY OF THE INVENTION

One technical problem to be solved by the present invention is to provide a guide plate for spine surgery. The guide plate has higher accuracy, it can more effectively assist operation, and can locate and burnish the screw insertion point, convenient for inserting the guide pin. The second technical problem to be solved by the present invention is to provide a production method of the aforesaid guide plate for spine surgery, the guide plate obtained by the production method has higher accuracy. The third technical problem to be solved by the present invention is to provide a usage of the aforesaid guide plate for spine surgery.

For these reasons, the guide plate for spinal surgery provided by the present invention, comprising a guide plate body, wherein the guide plate body includes a medial surface matching a back surface of a surgical segment centrum; a guide hole disposed on the guide plate body into which a guide pin can be inserted, a flange disposed on an upper side of the guide hole, the guide hole is provided on a guide block, an outer surface of the guide block is conical, and the guide plate body is provided with a drill hole corresponding to the guide block.

More particularly, wherein an inner side wall of the drill hole has an embedding groove, an outer side wall of the guide block has an insertion piece; wherein the insertion piece is embedded in the embedding groove after the guide block is embedded in the drill hole.

More particularly, wherein a sidewall of the embedding groove and/or drill hole is provided with a magnetic attraction mechanism A matching the guide block, a front end sidewall of the guide block and the insertion piece are provided with a magnetic attraction mechanism B matching the magnetic attraction mechanism A; when the guide block and insertion piece to are inserted into the drill hole and embedding groove respectively, the magnetic attraction mechanism A attracts the magnetic attraction mechanism B.

More particularly, wherein the guide plate body includes a lower plate and an upper plate, the medial surface is on the lower plate, the medial surface has an anti-slip layer, the upper plate and the lower plate abut on each other and are fixed,

More particularly, wherein the anti-slip layer is a silica gel material layer, the drainage channels are staggered on the silica gel material layer.

More particularly, the guide plate body is made of transparent material.

More particularly, the medial surface of the drill hole is provided with a metal bedding, a lower port section of the drill hole is circular, an upper part section of the drill hole is elliptic, square, triangular, pentagonal or polygonal, the sidewall of the guide block is provided with a magnet which can implement magnetic attraction to the metal bedding.

Besides, A production method of the guide plate for spine surgery defined in claim 1 includes the following steps:

A. performing preoperative CT image scanning for a patient to obtain the image data of target surgical segment centrum, and performing 3D reconstruction,

B. setting up the screw diameter, degerming the data of screw path in the guide plate according to 2D images in various directions,

C. designing the guide plate for spine surgery according to the data obtained in the above steps, and performing 3D printing.

More particularly, the method further obtaining CT image data of patient from PACS system, which are saved in *. Dcm format, and importing into Mimics software to generate the views in axial, coronal and sagittal directions, using Segment module function to select the segment region to be operated in Mimics;

B. simulating the insertion of pedicle screw, setting up the screw diameter, determining the screw path according to 2D images in various directions,

the segment to be operated and the simulated screw path are exported from Mimics and imported into Geomagic to select the guide plate region, and the region is thickened to make the sheet model,

the sheet model data generated in Geomagic is exported, and reimported into Mimics software to fabricate the guide plate model, the length and diameter of cylinder for simulating the screw path are adjusted to formulate the length, inside diameter and outside diameter of guide channel on the required guide plate model, and the Boolean function of Mimics software is used for Boolean operation of various guide plate components to make the required guide plate model;

C. making end product by 3D printing according to guide plate model data.

Moreover, a method for using the guide plate for spine including

A. removing the guide block on the guide plate;

B. placing the medial surface of the guide plate against the centrum adaptation surface, and using drill hole to locate the position of screw insertion point in the centrum back surface; then

C. embedding the guide block. into the drill hole, and using the guide hole on the guide block to accurately guide the guide pin for the insertion of the hollow screw.

Technical effects of the present invention:

1. The medial surface of guide plate of the present invention matches the surface of vertebral lamina, as the vertebra surface is in irregular shape, when the guide plate covers an adaptive region, the medial surface of guide plate abuts on the vertebra surface, it is unlikely to move under appropriate pressure.

2. In the present invention, the guide block can be taken out before the guide pin is inserted, the abrasive drilling bit burnishes the position the screw is to he driven in (i.e. where the drill hole is exposed), and then the guide block is inserted into the hole, convenient for the operator to insert the guide pin into the guide hole in the guide plate. As the diameter of the front end of drill hole is about 3 MM, and the diameter of guide hole is 1.8 MM, the aperture difference can be remedied by arranging the guide hole in the guide block, so that the entire operation can be completed more accurately and rapidly with the assistance of guide plate.

### BRIEF DESCRIPTION OF THE DRAWINGS

FIG. 1 is a three-dimensional structure diagram of the guide plate provided in the Embodiment 1 of the present invention;

FIG. 2 is a side view structural representation of FIG. 1;

FIG. 3 is a broken-out section view of guide hole in FIG. 1;

FIG. 4 is a split structure diagram of guide hole in FIG. 3;

FIG. 5 is a partial split structure section view of guide plate provided with insertion piece and embedding groove in FIG. 3;

FIG. 6 is a partial top view of drill hole of guide plate in FIG. 5;

FIG. 7 is a local structure section view of drill hole of guide plate is provided in the Embodiment 2 of the present invention;

FIG. 8 is a schematic plan of local structure of silica gel material layer on medial surface of guide plate in FIG. 7;

FIG. 9 is a local split structure diagram of guide plate provided in the Embodiment 3 of the present invention;

FIG. 10 is a partial top view of drill hole of guide plate in FIG. 9.

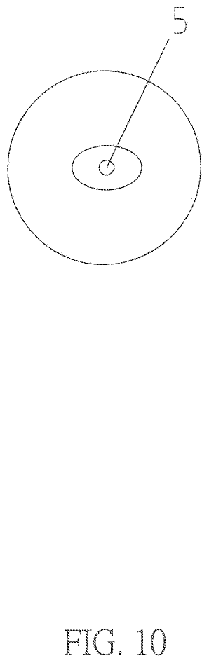

### DETAILED DESCRIPTION OF THE INVENTION

The present invention is further described below with attached figures and embodiments. The same part is represented by the same attached figure mark. Please note that the words “front”, “back”, “left”, “right”, “up” and “down” used in the description refer to directions in the attached figures. The words “bottom surface” and “top surface”, “inner” and “outer” refer to the directions toward or far from the geometric center of specific component.

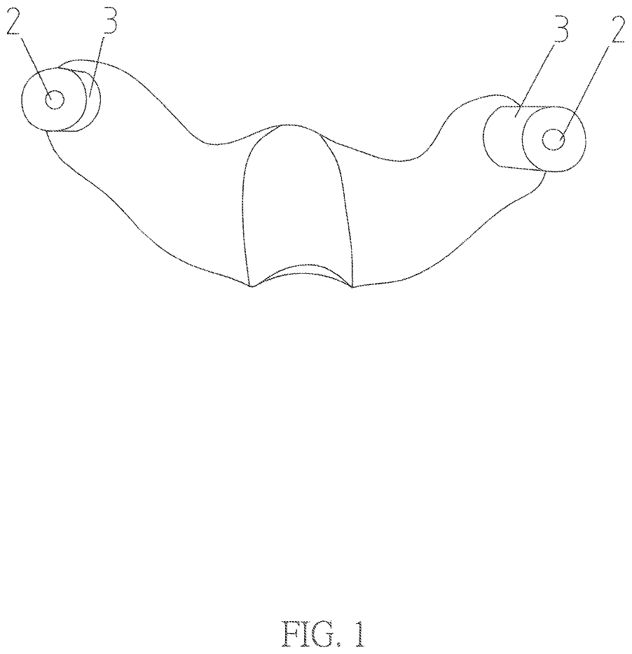

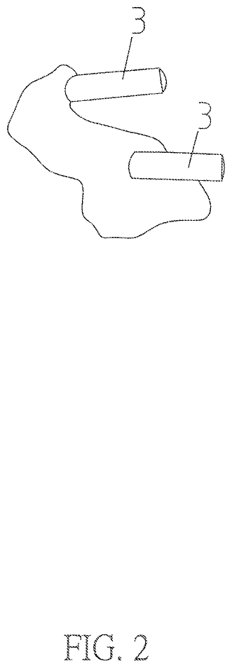

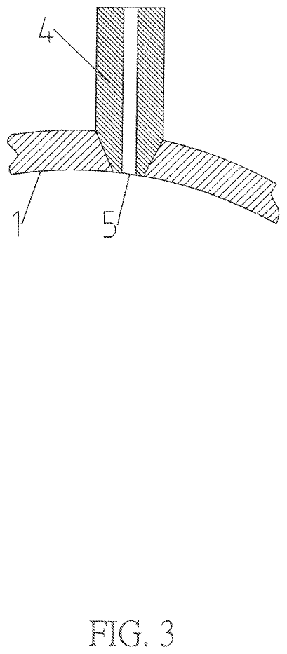

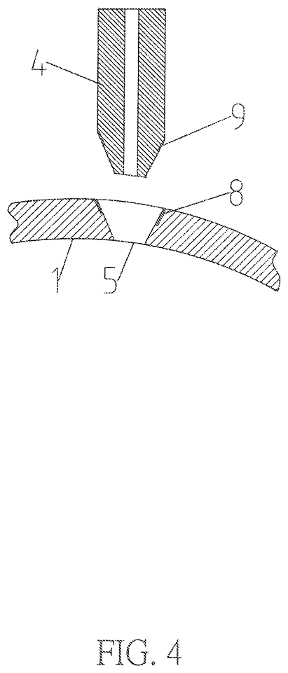

As shown in FIGS. 1-6, the guide plate for spine surgery provided in the Embodiment 1 of the present invention includes a guide plate body, the to guide plate body includes a medial surface matching the back surface of surgical segment centrum, the guide plate body is provided with a guide hole 2 over against the position the guide pin is to be inserted in, the upside of guide hole 2 has a flange 3. Said guide hole 2 is arranged in the guide block 4, the outer surface of guide block 4 is conical. Said guide plate body is provided with a drill hole 5 corresponding to said guide block 4. The drill hole 5 matches the outer surface of front end of said guide block 4, said guide block. 4 is embedded in the drill hole 5.

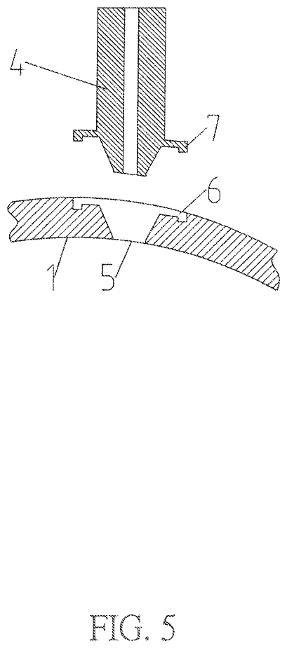

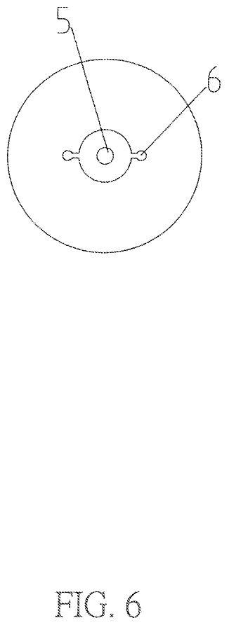

As shown in FIGS. 5-6, the inner side wall of said drill hole has an embedding groove 6, the outer side wall of said guide block has an insertion piece 7. When said guide block 4 is embedded in the drill hole 5, said insertion piece 7 is embedded in the embedding groove 6. The fitting structure of insertion piece 7 and embedding groove 6 is favorable for keeping stable setting of insertion piece 7. The sidewall of said embedding groove. 6 and/or drill hole 5 is provided with a magnetic attraction mechanism A8 matching the guide block. The front end sidewall of said guide block 4 and insertion piece 7 are provided with a magnetic attraction mechanism B9 matching the magnetic attraction mechanism A8. When said guide block 4 and insertion piece 7 are inserted into the drill hole 5 and embedding groove 6 respectively, said magnetic attraction mechanism A8 attracts magnetic attraction mechanism B9. The magnetic attraction mechanism further enhances the firmness and convenience of combination s of guide block 4 and guide plate body.

For observing the operation state, in the aforesaid Embodiment 1, the guide plate body is made of transparent material.

As shown in FIGS. 1-6, the production method of the aforesaid guide plate for spine surgery provided in the Embodiment 1 of the present invention includes the following steps;
A. perform CT image scanning for the patient to obtain the image data of target surgical segment centrum, and perform 3D reconstruction.
B. set up the screw diameter, determine the data of screw path in said guide plate according to 2D images in various directions.
C. design the guide plate for spine surgery according to the data obtained by Steps A and B, and perform 3D printing.

And further include the following steps:
A. obtain CT image data of cervical vertebrae from PACS system (scanning layer thickness is about 1 mm), which are saved in *.Dcm format, and imported into Mimics software to generate the views in axial, coronal and sagittal directions.

The Segment module function is used to select the segment region to be operated in Mimics.
B. simulate the insertion of pedicle screw, set up the screw diameter, determine the screw path according to 2D images in various directions,

The segment to be operated and the simulated screw path are exported from Mimics and imported into Geomagic to select the guide plate region, and the region is thickened to make the sheet model.

The sheet model data generated in Geomagic are exported, and reimported into Mimics software to build the guide plate model. The length and diameter of cylinder are simulated by adjusting the screw path, so as to formulate the length, inside diameter and outside diameter of guide channel is on the required guide plate model, and the Boolean function of Mimics software is used for Boolean operation of guide plate components, so as to fabricate the required guide plate model.
C. Make end product by 3D printing according to guide plate model data
   The aforesaid guide plate production method can be combined with the data of the bone surface to be operated, the guide plate can be fabricated more conveniently and accurately.

As shown in FIGS. 1-6, the usage of the guide plate for spine surgery provided in the Embodiment 1 of the present invention, A, the guide block 4 on said guide plate is removed; B, the medial surface of said guide plate adheres to the centrum adaptation surface, the position of screw insertion point in the centrum back surface is located by drill hole 5; C, said guide block 4 is embedded in the drill hole 5, the guide pin is accurately guided by the guide hole 2 in guide block 4, for inserting the hollow screw. The usage of the guide plate can effectively burnish the cortical bone surface of surgical centrum screw insertion point beforehand.

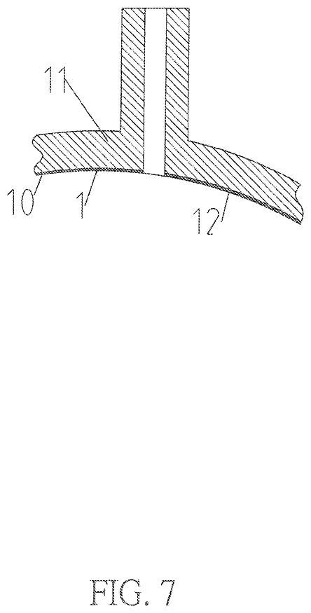

As shown in FIG. 7, the Embodiment 2 of the present invention is basically identical with Embodiment 1, the only difference is that said guide plate body includes a lower plate 10 and an upper plate 11, said medial surface is on the lower plate 10, said medial surface has an anti-slip layer. Said upper plate and said lower plate abut on each other and are fixed. The anti-slip layer is a silica gel material layer 12. The drainage channels 13 are staggered on the silica gel material layer 12. The silica gel material layer 12 in this embodiment can enhance the non-skid property of the joint of guide plate and bones, and the drainage channels 13 can discharge the residual liquid from the bone surface in time to avoid it influencing the non-skid property.

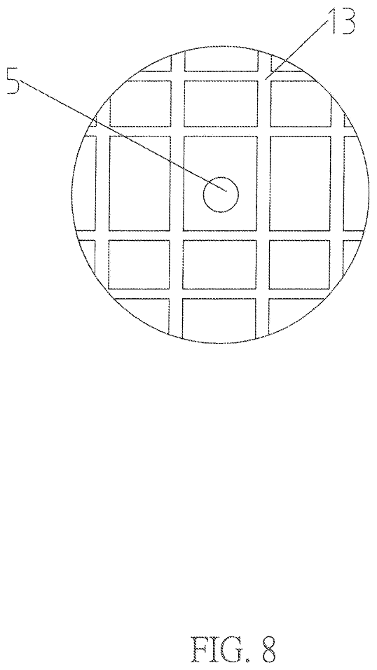

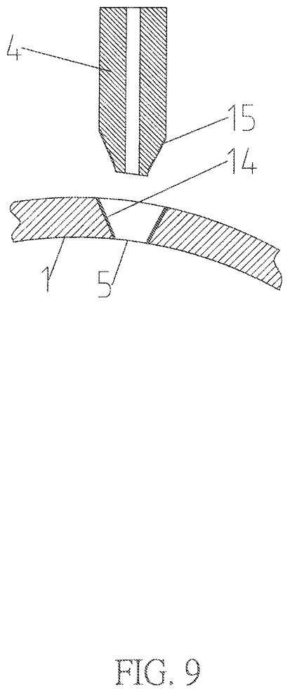

As shown in FIGS. 8-9, the Embodiment 3 of the present invention is basically identical with Embodiment 1, the only difference is that the medial surface of said drill hole is provided with a metal bedding 14, the is lower port section of said drill hole is circular, the upper part section of said drill hole is elliptic, square, triangular, pentagonal or polygonal. The sidewall of said guide block is provided with a magnet 15 which can implement magnetic attraction to said metal bedding. The metal bedding 14 can avoid the drilling bit directly impacting the plastic guide plate body when burnishing the cortical bone at the drill hole, so as to avoid plastic chips influencing the surgical effect.

We claim:

1. A guide plate for spine surgery comprising
a guide plate body,
wherein the guide plate body includes
a medial surface matching a back surface of a surgical segment centrum;
a guide hole disposed on the guide plate body into which a guide pin can be inserted,
a flange disposed on an upper side of the guide hole,
the guide hole is provided on a guide block, an outer surface of the guide block is conical, and the guide plate body is provided with a drill hole corresponding to the guide block.

2. The guide plate for spine surgery defined in claim 1, wherein an inner side wall of the drill hole has an embedding groove, an outer side wall of the guide block has an insertion piece; wherein the insertion piece is embedded in the embedding groove after the guide block is embedded in the drill hole.

3. The guide plate for spine surgery defined in claim 2, wherein a sidewall of the embedding groove and/or drill hole is provided with a magnetic attraction mechanism A matching the guide block, a front end sidewall of the guide block and the insertion piece are provided with a magnetic attraction mechanism B matching the magnetic attraction mechanism A; when the guide block and insertion piece are inserted into the drill hole and embedding groove respectively, the magnetic attraction mechanism A attracts the magnetic attraction mechanism B.

4. The guide plate for spine surgery defined in claim 2, wherein the guide plate body includes a lower plate and an upper plate, the medial surface is on the lower plate, the medial surface has an anti-slip layer, the upper plate and the lower plate abut on each other and are fixed.

5. The guide plate for spine surgery defined in claim 4, wherein the anti-slip layer is a silica gel material layer, the drainage channels are staggered on the silica gel material layer.

6. The guide plate for spine surgery defined in claim 1, wherein the guide plate body is made of transparent material.

7. The guide plate for spine surgery defined in claim 1, wherein the medial surface of the drill hole is provided with a metal bedding, a lower port section of the drill hole is circular, an upper part section of the drill hole is elliptic, square, triangular, pentagonal or polygonal, the sidewall of the guide block is provided with a magnet which can implement magnetic attraction to the metal bedding.

8. A production method of the guide plate for spine surgery defined in claim 1 includes the following steps:
A. performing preoperative CT image scanning for a patient to obtain the image data of target surgical segment centrum, and performing 3D reconstruction,
B. setting up the screw diameter, degerming the data of screw path in the guide plate according to 2D images in various directions,
C. designing the guide plate for spine surgery according to the data obtained in the above steps, and performing 3D printing.

9. The production method of the guide plate for spine surgery defined in

8. , including the following step:
A. obtaining CT image data of patient from PACS system, which are saved in *. Dcm format, and importing into Mimics software to generate the views in axial, coronal and sagittal directions,
using Segment module function to select the segment region to be operated in Mimics;
B. simulating the insertion of pedicle screw, setting up the screw diameter, determining the screw path according to 2D images in various directions,
the segment to be operated and the simulated screw path are exported from Mimics and imported into Geomagic to select the guide plate region, and the region is thickened to make the sheet model,
the sheet model data generated in Geomagic is exported, and reimported into Mimics software to fabricate the guide plate model, the length and diameter of cylinder for simulating the screw path are adjusted to formulate the length, inside diameter and outside diameter of guide channel on the required guide plate model, and the Boolean function of Mimics software is used for Boolean operation of various guide plate components to make the required guide plate model;
C. making end product by 3D printing according to guide plate model data.

10. A method for using the guide plate for spine surgery stated in claim 1, including
A. removing the guide block on the guide plate;
B. placing the medial surface of the guide plate against the centrum adaptation surface, and using drill hole to locate the position of screw insertion point in the centrum back surface; then
C. embedding the guide block into the drill hole, and using the guide hole on the guide block to accurately guide the guide pin for the insertion of the hollow screw.
